(19) United States
(12) Patent Application Publication
(10) Pub. No.: US 2019/0110671 A1
(43) Pub. Date: Apr. 18, 2019

(54) ILLUMINATING DEVICE INCLUDING NARROW BAND LIGHT SOURCES

(71) Applicant: OLYMPUS CORPORATION, Tokyo (JP)

(72) Inventor: Bakusui DAIDOJI, Hachioji-shi (JP)

(73) Assignee: OLYMPUS CORPORATION, Tokyo (JP)

(21) Appl. No.: 16/214,570

(22) Filed: Dec. 10, 2018

Related U.S. Application Data
(63) Continuation of application No. PCT/JP2016/069877, filed on Jul. 5, 2016.

(51) Int. Cl. A61B 1/06; H05B 33/08; A61B 1/00; A61B 1/07

(52) U.S. Cl. CPC A61B 1/063; H05B 33/08; A61B 1/00006; A61B 1/07; A61B 1/0653; A61B 1/0684; A61B 1/0669; A61B 1/0638

(57) ABSTRACT

An illuminating device includes narrow band light sources including same-color narrow band light sources each configured to emit same-color narrow band light. The same-color narrow band light sources are divided into groups each including at least one narrow band light source. The illuminating device also includes a same-color light source controller configured to control the same-color narrow band light sources and perform light control of emitted light from the same-color narrow band light sources by increasing or decreasing, for each of the groups, an emitted-light quantity of the same-color narrow band light sources within a predetermined reference period, by a unit gradation in a predetermined order.

### CROSS-REFERENCE TO RELATED APPLICATIONS

This application is a Continuation Application of PCT Application No. PCT/JP2016/069877, filed Jul. 5, 2016, the entire contents of which are incorporated herein by reference.

### BACKGROUND OF THE INVENTION

### 1. Field of the Invention

The present invention relates to an illuminating device including narrow band light sources.

### 2. Description of the Related Art

In recent years, an illuminating device including a narrow band light source such as a laser light source has been proposed. An illuminating device using a laser light source has a problem in which an interference pattern (speckle) occurs due to high coherence of laser light.

Jpn. Pat. Appln. KOKAI Publication No. 2002-95634 discloses an illuminating device including same-color laser light sources with different wavelengths each other in order to reduce speckles. In this illuminating device, using same-color laser light sources with different wavelengths each other averages speckles to reduce the speckles as compared with an illuminating device using a single laser light source.

### BRIEF SUMMARY OF THE INVENTION

An illuminating device includes narrow band light sources including same-color narrow band light sources each configured to emit same-color narrow band light. The same-color narrow band light sources are divided into groups each including at least one narrow band light source. The illuminating device also includes a light source controller configured to control the narrow band light sources. The light source controller includes a same-color light source controller configured to control the same-color narrow band light sources and perform light control of emitted light from the same-color narrow band light sources by increasing or decreasing, for each of the groups, an emitted-light quantity of the same-color narrow band light sources within a predetermined reference period, by a unit gradation in a predetermined order.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 schematically shows a configuration of an endoscope including an illuminating device according to a present embodiment.

FIG. 2 shows an example of spectroscopic characteristics of color filters.

FIG. 3 is a diagram of color space coordinates of a CIE 1976 L*u*v* color system.

FIG. 4 shows driving pulses of same-color laser light sources according to a light control method of the present embodiment.

FIG. 5 shows driving pulses of the same-color laser light sources according to a light control method of a modification.

FIG. 6 shows same-color laser light sources connected in series and included in the same group.

FIG. 7 shows driving pulses of the same-color laser light sources according to a light control method of another modification.

FIG. 8 shows driving pulses of the same-color laser light sources according to a light control method of yet another modification.

### DETAILED DESCRIPTION OF THE INVENTION

### First Embodiment

[Configuration]

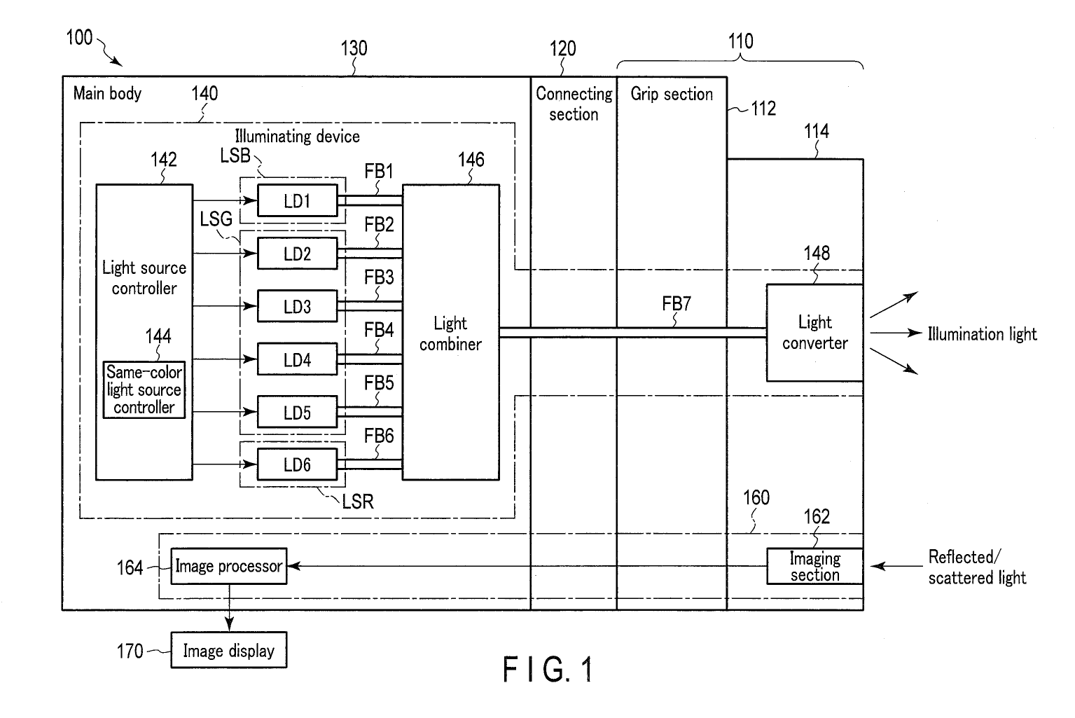

FIG. 1 schematically shows a configuration of an endoscope including an illuminating device according to the present embodiment.

An endoscope 100 includes a scope 110, a main body 130, and a connecting section 120 configured to connect the scope 110 and the main body 130. The scope 110 has a grip section 112 for an operator to grip, and an insertion section 114 extending from the grip section 112. The insertion section 114 is a hollow elongated flexible member to be inserted into the internal space of an observation object such as a lumen, etc. The grip section 112 is provided with a control section such as a knob, a lever, a dial, etc., for operating the insertion section 114.

The endoscope 100 also includes an illuminating device 140 configured to emit illumination light, and an imaging device 160 configured to image an observation object. An image display 170 configured to display an image of an observation object is connected to the main body 130 of the endoscope 100.

[Illuminating Device 140]

The illuminating device 140 comprises: laser light sources LD1 to LD6 as narrow band light sources; a light source controller 142 configured to control the laser light sources LD1 to LD6; optical fibers FB1 to FB6 as light guides configured to guide laser light emitted from the laser light sources LD1 to LD6; a light combiner 146 configured to combine the laser light guided by the optical fibers FB1 to FB6; an optical fiber FB7 as a light guide configured to guide laser light combined by the light combiner 146; and a light converter 148 configured to convert the laser light guided by the optical fiber FB7 into a illumination light and emits this illumination light. For example, the laser light sources LD1 to LD6, the light source controller 142, and the light combiner 146 are provided inside the main body 130, and the light converter 148 is provided at a distal end of the insertion section 114.

According to another viewpoint, the illuminating device 140 includes a blue light source section LSB configured to emit blue light, a green light source section LSG configured to emit green light, and a red light source section LSR configured to emit red light. The blue light source section LSB includes the laser light source LD1, the green light source section LSG includes the laser light sources LD2 to LD5, and the red light source section LSR includes the laser light source LD6.

[Narrow Band Light Sources (Laser Light Sources LD1 to LD6)]

Each of the laser light sources LD1 to LD6 may comprise, for example, a laser diode. Examples of the rated light quantity and wavelength are described below.

The laser light source LD1 is a blue laser diode, and emits light having a center wavelength of 445 nm. The rated light quantity of the laser light source LD1 is 3 W.

The laser light source LD2 is a green laser diode, and emits light having a center wavelength of 525 nm. The rated light quantity of the laser light source LD2 is 1 W.

The laser light source LD3 is a green laser diode, and emits light having a center wavelength of 525 nm. The rated light quantity of the laser light source LD3 is 1 W.

The laser light source LD4 is a green laser diode, and emits light having a center wavelength of 520 nm. The rated light quantity of the laser light source LD4 is 1 W.

The laser light source LD5 is a green laser diode, and emits light having a center wavelength of 530 nm. The rated light quantity of the laser light source LD5 is 1 W.

The laser light source LD6 is a red laser diode, and emits light having a center wavelength of 635 nm. The rated light quantity of the laser light source LD6 is 3 W.

Regarding the laser light sources LD1 to LD6, the laser light sources LD2 to LD5 are the same-color narrow band light sources (same-color laser light sources) each configured to emit the same-color narrow band light (laser light). With respect to a predetermined color, if the desired quantity of emitted light cannot be obtained using a single laser light source, using same-color laser light sources is an effective manner. Further, use of same-color laser light sources enables speckles to be averaged and reduced in that color.

The “same-color narrow band light sources (same-color laser light sources)” in the present invention are defined as narrow band light sources (laser light sources) each configured to emit the same-color narrow band light (laser light). Furthermore, the “same-color narrow band light (laser light)” in the present invention is defined as narrow band light (laser light) included in the same color range for, for example, three color ranges of a blue range, a green range, and a red range. Here, the blue range, the green range, and the red range may be obtained by dividing, for example, a visible light wavelength range from 400 to 700 nm into three ranges, and allocating them as a blue range, a green range, and a red range sequentially from the shortest wavelength range. Furthermore, for example, these wavelength ranges may be obtained by dividing a visible light wavelength range from 400 to 700 nm equally into three ranges with overlap of 20 nm between the adjacent ranges, and setting the blue range to a wavelength range from 400 to 510 nm, the green range to a wavelength range from 490 to 610 nm, and the red range to a wavelength range from 590 to 700 nm. In doing so, the wavelength range of 400 nm or less and the wavelength range of 700 nm or more may be allocated as the blue range and the red range, respectively.

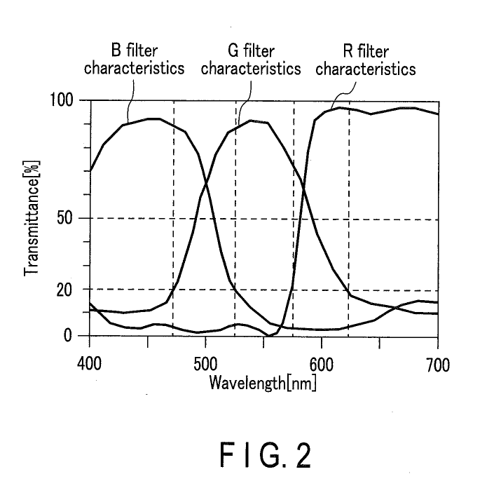

Furthermore, if the imaging device 160 has, for example, an imaging section having primary color filters, the blue range, the green range, and the red range may be defined based on the spectroscopic characteristics of the primary color filters. In this case, the same-color narrow band light (laser light) may be defined as narrow band light (laser light) included in a wavelength range equal to or higher than a predetermined transmittance in each of the color filters. FIG. 2 shows an example of spectroscopic characteristics of color filters. In those color filters, for example, in filter characteristics of each color (each of R filter characteristics, G filter characteristics, and B filter characteristics), a wavelength range having a transmittance of 20% or more is defined as each color range. That is, in the color filters having the spectroscopic characteristics shown in FIG. 2, the blue region corresponds to a wavelength range from 400 to 525 nm, the green range corresponds to a wavelength range from 470 to 625 nm, and the red range corresponds to a wavelength range from 570 to 700 nm. As shown in FIG. 2, the filter characteristics of each color hardly include a wavelength range in which the transmittance is zero, and has the transmittance of several % to 10% or so in a broad range of the visible light. The transmittance of several % to 10% or so is regarded as a negligible level in capturing a color image. Therefore, there is no problem even if a color range is defined based on a range in which the transmittance is 20% or higher.

Also, in specific light observation in which a specific substance is emphasized and observed by illuminating illumination light in a specific wavelength range such as NBI (Narrow Band Imaging), for example, light included in each of wavelength ranges necessary for specific light observation may be regarded as the same color. That is, the same-color narrow band light (laser light) used for illumination of specific light observation may be defined as narrow band light (laser light) included in each of the wavelength ranges necessary for specific light observation. In NBI, for example, rays of narrow band light (laser light) within the wavelength range from 390 to 445 nm may be regarded as the same color, and rays of narrow band light (laser light) within the wavelength range from 530 to 550 nm may be regarded as the same color.

Furthermore, for example, in fluorescent observation that irradiates a specific substance with excitation light to observe fluorescent light emitted from the specific substance, rays of light included in an excitation wavelength band of the specific substance as a fluorescent observation object may be regarded as the same color. That is, the same-color narrow band light (laser light) to be used for illumination of fluorescent observation may be defined as narrow band light (laser light) included in the excitation wavelength band of a specific substance.

For example, rays of narrow band light (laser light) having central wavelengths within the range of ±50 nm may be defined as the same color. It is preferable that rays of the same-color narrow band light (laser light) have center wavelengths that fall within the range of ±20 nm, and such rays of the same-color narrow band light (laser light) show roughly equivalent color reproducibility. Furthermore, it is more preferable that rays of the same-color narrow band light (laser light) have center wavelengths that fall within the range of ±10 nm.

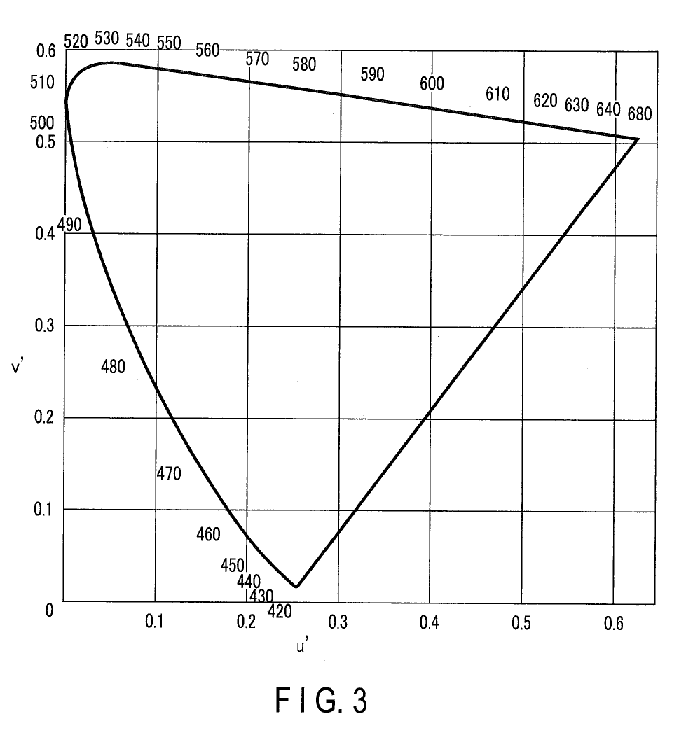

Furthermore, for example, the same color may be defined based on a color difference of reflected light with respect to a representative object, such as a white chart. For example, rays of narrow band light (laser light) in which distance ([(u*)²⁺(v*)²]^{1/2}) between their color space coordinates in the CIE 1976 L*u*v* color system shown in FIG. 3 is 0.2 or less are defined as the same color. Narrow band light (laser light) within this range appears to be roughly the same color. Furthermore, for rays of the same-color narrow band light (laser light), it is more preferable that a distance ([(u*)²+(v*)²]^{1/2}) between their color space coordinates be 0.1 or less.

[Light Guides (Optical Fibers FB1 to FB6)]

Each of the optical fibers FB1 to FB6 has an end optically connected to each of the laser light sources LD1 to LD6 and the other end optically connected to the light combiner 146, so as to guide laser light emitted from the laser light sources LD1 to LD6 to the light combiner 146.

Each optical fibers FB1 to FB6 comprises a single-wire fibers each having a core diameter of, for example, several tens of μm to several hundreds of μm. An optical coupling lens (not shown in the drawings) for converging laser light emitted from each of the laser light sources LD1 to LD6 to couple it to the optical fibers FB is provided in each of spaces between the laser light sources LD1 to LD6 and the optical fibers FB1 to FB6.

[Light Combiner 146]

The light combiner 146 is a light combiner with six inputs and a single output, in which the optical fibers FB1 to FB6 are optically connected to the ends of the six inputs, and the optical fiber FB7 is optically connected to the end of the single output. The light combiner 146 comprises, for example, an optical fiber combiner. The light combiner 146 combines laser light guided by the optical fibers FB1 to FB6, to output them to the single optical fiber FB7. Furthermore, for example, the light combiner 146 may comprise a spatial optical system having a lens and dichroic mirrors.

[Light Guide (Optical Fiber FB7)]

The optical fiber FB7 has an end optically connected to the light combiner 146 and the other end optically connected to the light converter 148, so as to guide laser light emitted from the light combiner 146 to the light converter 148.

The optical fiber FB7 comprises a single-wire fiber having a core diameter of, for example, several tens of μm to several hundreds of μm. Furthermore, the optical fiber FB7 may, for example, comprise a bundle fiber comprising (several hundreds to several thousands of) optical fibers.

[Light Converter 148]

The light converter 148 includes, for example, a diffusing member configured to diffuse laser light guided by the optical fiber FB7. Light diffusion has the effect of broadening the light distribution of illumination light, and the effect of disturbing a phase of laser light to reduce the coherence, thereby reducing speckles. The diffusing member is, for example, a structure in which metal particles with a high reflectance are dispersed in resin or glass. For example, the light converter 148 may include a lens for broadening the light distribution.

[Light Source Controller 142]

The light source controller 142 controls the driving of each of the laser light sources LD1 to LD6. The light source controller 142 is capable of controlling ON/OFF, driving current, and a driving method (continuous wave oscillation (CW), pulse driving, etc.) of the laser light sources LD1 to LD6 independently of each other, by outputting a light-source driving signal to each of the laser light sources LD1 to LD6.

In addition, the light source controller 142 has a same-color light source controller 144 configured to control the same-color laser light sources, that is, the green laser light sources LD2 to LD5. The same-color light source controller 144 is capable of controlling ON/OFF, driving current, and a driving method of the laser light sources LD2 to LD5 independently of each other.

[Imaging Device 160]

The imaging device 160 has an imaging section 162 provided at the distal end of the insertion section 114 and an image processor 164 provided inside the main body 130.

[Imaging Section 162]

The imaging section 162 receives reflected/scattered light of illumination light from an observation object to acquire an optical image of the observation object illuminated by the illuminating device 140. The imaging section 162 also generates an electric imaging signal corresponding to the acquired optical image, and outputs the generated imaging signal to the image processor 164. The imaging section 162 comprises, for example, a CCD or a CMOS. The imaging section 162 includes, for example, RGB color filters as shown in FIG. 2, on the front face of the light receiver. The imaging section 162 acquires each of optical images separately for each of three wavelength ranges including the red range, the green range, and the blue range, generating an R imaging signal, a G imaging signal, and a B imaging signal. The imaging section 162 repeatedly performs imaging in a predetermined imaging frame period Tf. Also, the imaging section 162 may, for example, be a monochrome imager having no color filter. In this case, the blue light source section LSB, the green light source section LSG, and the red light source section LSR sequentially emit blue light, green light, and red light, respectively, at different timings. The imaging section 162 sequentially receives reflected/scattered light for blue light, green light, and red light, at different timings, so as to generate imaging signals therefrom. Then the image processor 164 performs RGB assignment processing.

[Image Processor 164]

The image processor 164 generates an image of an observation object by known image processing for an imaging signal output from the imaging section 162. If the imaging section 162 is a monochrome imager, RGB assignment processing is first performed for imaging signals sequentially generated at different timings, and then an image of an observation object is generated.

[Image Display 170]

The image display 170 displays an image of the observation object, which is generated by the image processor 164. The image display 170 comprises, for example, a monitor such as a liquid crystal display.

[Light Control Method of Light Emitted from Laser Light Sources LD1 to LD6]

For the laser light sources LD1 to LD6, the light source controller 142 controls the light quantity ratio of emitted light among the blue laser light source LD1, the green laser light sources LD2 to LD5, and the red laser light source LD6 so that, for example, the illumination light is to be white light.

Herein, white light indicates a color that reproduces a color of broadband illumination light, such as xenon lamp. Alternatively, white light is of a color that reproduces a color of an observation object when it is irradiated with broadband illumination light, such as xenon lamp. More specifically, white light is defined using, for example, chromaticity coordinates, a correlated color temperature, or a color difference from a black body locus. For example, it is defined as, in the chromaticity coordinates, a color within the ranges (x=0.2-0.4, y=0.2-0.4), (x=0.4-0.5, y=0.35-0.45), or in the correlated color temperature, a color of the range from 2000 to 100000K, and a color of the range in which the color difference (duv) from the black body locus is ±0.1 or less. Furthermore, white light may be defined in consideration of the spectral sensitivity of the imaging section 162. For example, white light may be defined as above, using the chromaticity coordinates or correlated color temperature calculated for the spectrum obtained by multiplying the spectrum of illumination light with the spectral sensitivity of the imaging section 162.

[Light Control Method of Emitted Light from Same-Color Laser Light Sources LD2 to LD5]

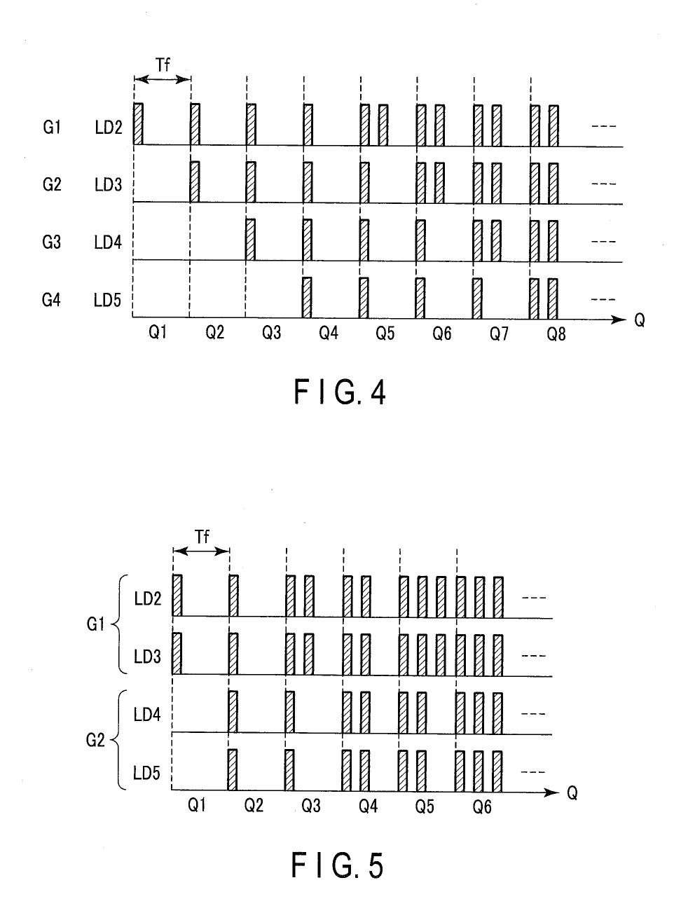

Hereinafter, an example of a light control method of emitted light from the same-color laser light sources LD2 to LD5. FIG. 4 shows driving pulses of the same-color laser light sources LD2 to LD5 according to a light control method of the present embodiment. FIG. 4 shows the case where the emitted-light quantity Q of all the green laser light sources LD2 to LD5 is increased by a unit gradation.

The laser light sources LD2 to LD5 are divided into groups each including at least one laser light source. In the present embodiment, all the same-color laser light sources LD2 to LD5 are respectively divided into different groups so that, for example, the laser light sources LD2 is in a group G1, the laser light source LD3 is in a group G2, the laser light source LD4 is in a group G3, and the laser light source LD5 is in a group G4.

The same-color light source controller 144 performs light control of emitted light from the same-color laser light sources LD2 to LD5 by increasing or decreasing, for each of the groups, the emitted-light quantity Q of the same-color laser light sources LD2 to LD5 within a predetermined reference period. The predetermined reference period is, for example, the imaging frame period Tf of the imaging section 162. The same-color light source controller 144 increases or decreases the emitted-light quantity Q by increasing or decreasing, for each of the groups, light emission times of the same-color laser light sources LD2 to LD5 within the imaging frame period Tf. In the present embodiment, the same-color light source controller 144 increases or decreases the emitted-light quantity Q by increasing or decreasing, for each of the groups, the number of pulses of the same-color laser light sources LD2 to LD5 within the imaging frame period Tf in a predetermined order.

In the state with the light quantity Q1, one pulse of the laser light source LD2 in the group G1 is emitted within the imaging frame period Tf.

In the next state with the light quantity Q2, one pulse of the laser light source LD3 in the group G2 is added with respect to the state with the light quantity Q1, within the imaging frame period Tf.

In the next state with the light quantity Q3, one pulse of the laser light source LD4 in the group G3 is added with respect to the state with the light quantity Q2, within the imaging frame period Tf.

In the next state with the light quantity Q4, one pulse of the laser light source LD5 in the group G4 is added with respect to the state with the light quantity Q3, within the imaging frame period Tf.

In the next state with the light quantity Q5, one pulse of the laser light source LD2 in the group G1 is added with respect to the state with the light quantity Q4, within the imaging frame period Tf.

Subsequently, a pulse is added in a similar manner.

If the emitted-light quantity Q of all the green laser light sources LD2 to LD5 is decreased by a unit gradation, control is performed in the reverse order of the above.

Furthermore, in the present embodiment, the same-color laser light sources LD2 to LD5 are laser light sources having substantially equal characteristics. In addition, peak light quantities of pulses and single pulse light emission times are also set substantially equal.

The same-color light source controller 144 controls the same-color laser light sources LD2 to LD5 so that a difference in light emission time among the same-color laser light sources LD2 to LD5 included in different groups within the imaging frame period Tf falls within a predetermined unit of light emission time. The same-color light source controller 144 causes the same-color laser light sources LD2 to LD5 to emit at least one pulse within the imaging frame period Tf and to increase or decrease the number of pulses, performing the light control. The predetermined unit of light emission time is a single pulse light emission time as a light emission time for the one pulse. Within the imaging frame period Tf, a difference in light emission time among the laser light sources LD2 to LD5 included in different groups corresponds to zero or the single pulse light emission time.

By performing control in this manner, the number of gradations of all of the same-color laser light sources LD2 to LD5 can be increased as compared with the case where all of the same-color laser light sources LD2 to LD5 are controlled in the same manner. Furthermore, by performing control in this manner, while the number of gradations is maintained, as many rays of the same-color light as possible are emitted in a state with a predetermined light quantity.

The same-color laser light sources LD2 to LD5 are the same in color, but are separate individuals. Thus, strictly speaking, phases and spectra of emitted light are different. Therefore, in illumination light obtained by combining rays of the same-color laser light, speckles can be reduced by averaging them as many as the same-color laser light sources LD2 to LD5. It is known that the effect of averaging speckles in N rays of laser light that are independent of each other and have the same light quantity is proportional to the reciprocal (N^{-1/2}) of the square root of N. By setting the peak light quantity of a pulse and the light emission time to be substantially equal as described above, rays of the same-color laser light, subjected to averaging, become substantially equal in emitted light quantity, so that the averaging effect is increased. Herein, “being substantially equal in peak light quantity of a pulse” means, for example, that a difference in peak light quantity between the light sources is equal to or less than a minimum peak light quantity gradation to be described later. In addition, “being substantially equal in emission time of a pulse” means, for example, that a difference in light emission time between the light sources is equal to or less than a single pulse light emission time to be described later.

The light control method described above, shown in FIG. 4, obtains the effect of averaging speckles in laser light in the state with the light quantity Q2, obtains a better effect of averaging speckles in laser light in the state with the light quantity Q3, and obtains an even better effect of averaging speckles in laser light in the state with the light quantity Q4 and the subsequent states.

The peak light quantity of a pulse is set within a range of the light quantity that is, for example, a multi-oscillation region, except for the case where the particularly small light quantity is required. In a multi-mode laser light source, as the peak light quantity (peak driving current) is increased, an oscillation mode increases and the spectrum width increases. As the spectrum width increases, the coherence decreases, and speckles are reduced. For example, a region in which the peak light quantity is 100 mW or more can be regarded as a multi-oscillation region in which an oscillation mode increases.

Furthermore, it is preferable that the same-color laser light sources LD2 to LD5 be substantially equal in peak light quantity. Since an increase and a decrease in the light quantity become substantially equal between light control steps, the light control is simplified.

Furthermore, the light emission time of a single pulse is set to a time during which a stable pulse waveform can be generated in the laser light sources or the same-color light source controller 144, for example. For example, the light emission time of a single pulse is set to several μs to several tens of μs.

In the present embodiment, the number of the same-color laser light sources LD2 to LD5 is four; however, the number of the same-color laser light sources is not limited thereto. For example, the number of the same-color laser light sources may be two, or five or more.

In the present embodiment, the same-color laser light sources LD2 to LD5 are equal in rated light quantity; however this is not a limitation.

Furthermore, the waveform of a pulse is not limited to the rectangular waveform shown in FIG. 4, but may be a multistage pulse, a triangular wave, or the like. In this respect, although in order to further reduce speckles, a high frequency wave may be superimposed on a driving current of a laser light source, the high frequency waveform in this case is not regarded as a pulse.

### Modification 1

Hereinafter, a modification of a light control method of emitted light from the same-color laser light sources LD2 to LD5 will be described. FIG. 5 shows driving pulses of the same-color laser light sources LD2 to LD5 according to the light control method of the present modification. As in FIG. 4, FIG. 5 shows the case where the emitted-light quantity Q of all the green laser light sources LD2 to LD5 is increased by a unit gradation.

In the present modification, the same-color laser light sources LD2 to LD5 are divided into two different groups so that the laser light sources LD2 and LD3 are in the group G1, and the laser light sources LD4 and LD5 are in the group G2. That is, in the present modification, laser light sources are included in the same group.

In the present modification also, the same-color light source controller 144 performs the light control of emitted light from the same-color laser light sources LD2 to LD5 by increasing or decreasing, for each of the groups, the emitted-light quantity Q from the same-color laser light sources LD2 to LD5 within a predetermined reference period. The predetermined reference period is, for example, the imaging frame period Tf of the imaging section 162. The same-color light source controller 144 increases or decreases the emitted-light quantity Q by increasing or decreasing, for each of the groups, light emission times of the same-color laser light sources LD2 to LD5 within the imaging frame period Tf. The same-color light source controller 144 increases or decreases the emitted-light quantity Q by increasing or decreasing, for each of the groups, the number of pulses of the same-color laser light sources LD2 to LD5 within the imaging frame period Tf in a predetermined order.

In the state with the light quantity Q1, one pulse of each of the laser light sources LD2 and LD3 in the group G1 is emitted within the imaging frame period Tf.

In the next state with the light quantity Q2, one pulse of each of the laser light sources LD4 and LD5 in the group G2 is added with respect to the state with the light quantity Q1, within the imaging frame period Tf.

In the next state with the light quantity Q3, one pulse of each of the laser light sources LD2 and LD3 in the group G1 is added with respect to the state with the light quantity Q2, within the imaging frame period Tf.

In the next state with the light quantity Q4, one pulse of each of the laser light sources LD4 and LD5 in the group G2 is added with respect to the state with the light quantity Q3, within the imaging frame period Tf.

Subsequently, a pulse is added in a similar manner.

If the required number of gradations is less than those shown in FIG. 4, light control may be performed by grouping the laser light sources as shown in FIG. 5. In this case, although the number of gradations is decreased as compared to the case shown in FIG. 4, since at least two rays of the same-color laser are emitted in all the light-quantity states, the averaging effect can be stably obtained for speckles. In addition, compared to the state with the light quantity Q1, the effect of averaging speckles is higher in the state with the light quantity Q2 and the subsequent states.

Within the imaging frame period Tf, the same-color light source controller 144 controls the same-color laser light sources LD2 to LD5 included in the same group so that the same-color laser light sources included in the same group are substantially equal in emitted-light quantity. In this case, the laser light source LD2 and the laser light source LD3 included in the group G1 are substantially the same in emitted-light quantity, whereas the laser light source LD4 and the laser light source LD5 included in the group G2 are substantially the same in emitted-light quantity. In this manner, speckles are effectively averaged in the same-color narrow band light sources included in the same group.

Furthermore, it is preferable that the numbers of the same-color laser light sources LD2 and LD3, and LD4 and LD5 included in each group are equal. Since an increase and a decrease in light quantity are substantially equal among light control steps, the light control is simplified. If the numbers of the same-color laser light sources LD2 and LD3, and LD4 and LD5 included in each group are not equal, it is preferable that a difference in number between the same-color laser light sources LD2 and LD3, and LD4 and LD5 be one or less.

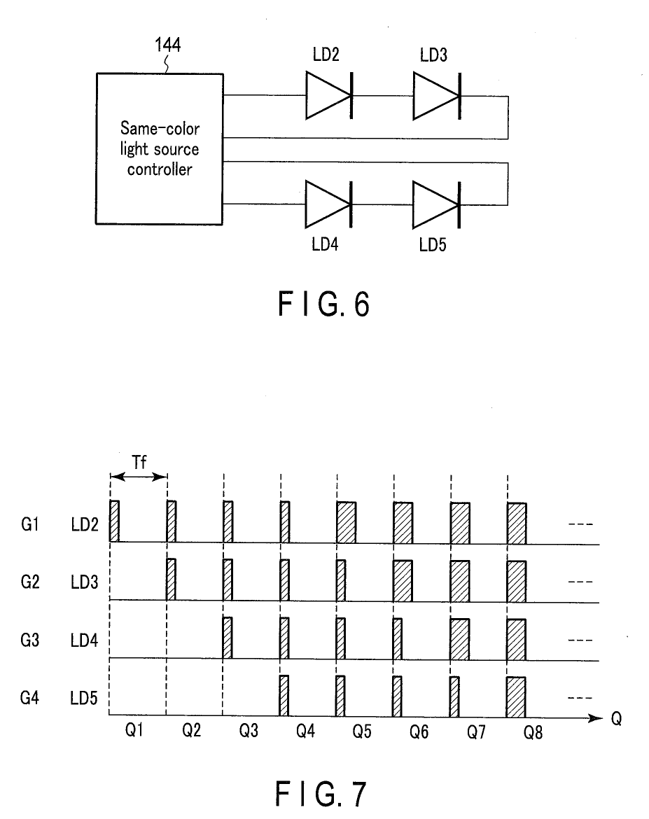

For example, the same-color light source controller 144 may perform control by synchronizing emission timings of the same-color laser light sources LD2 and LD3, and LD4 and LD5 included in the same groups. For example, it may be configured that the same-color light source controller 144 performs control by supplying driving currents to the same-color laser light sources LD2 to LD5, and as shown in FIG. 6, the same-color laser light sources LD2 and LD3, and LD4 and LD5 included in the same groups are connected in series.

### Modification 2

Hereinafter, another modification of a light control method of emitted light from the same-color laser light sources LD2 to LD5 will be described. FIG. 7 shows driving pulses of the same-color laser light sources LD2 to LD5 according to the light control method of the present modification. As in FIG. 4, FIG. 7 shows the case where the emitted-light quantity Q of all the green laser light sources LD2 to LD5 is increased by a unit gradation.

In the present modification also, all the same-color laser light sources LD2 to LD5 are respectively divided into different groups so that, for example, the laser light sources LD2 is in the group G1, the laser light source LD3 is in the group G2, the laser light source LD4 is in the group G3, and the laser light source LD5 is in the group G4.

Also in the present modification, the same-color light source controller 144 performs the light control of emitted light from the same-color laser light sources LD2 to LD5 by increasing or decreasing, for each of the groups, the emitted-light quantity Q of all the same-color laser light sources LD2 to LD5 within a predetermined reference period. The predetermined reference period is, for example, the imaging frame period Tf of the imaging section 162. The same-color light source controller 144 increases or decreases the emitted-light quantity Q by increasing or decreasing, for each of the groups, light emission times of the same-color laser light sources LD2 to LD5 within the imaging frame period Tf. However, in the present modification, the same-color light source controller 144 increases or decreases the emitted-light quantity Q by increasing or decreasing, for each of the groups, a pulse width of the same-color laser light sources LD2 to LD5 within the imaging frame period Tf.

The state with the light quantity Q1 to the state with the light quantity Q4 are the same as those in the first embodiment.

In the next state with the light quantity Q5, within the imaging frame period Tf, a pulse width of the laser light source LD2 in the group G1 is wider by a minimum pulse width gradation with respect to the state with the light quantity Q4.

In the next state with the light quantity Q6, within the imaging frame period Tf, a pulse width of the laser light source LD3 in the group G2 is wider by the minimum pulse width gradation with respect to the state with the light quantity Q5.

Subsequently, a pulse width is widened in a similar manner.

As described above, the same-color light source controller 144 controls the same-color laser light sources LD2 to LD5 so that a difference in light emission time among the same-color laser light sources LD2 to LD5 included in different groups within the imaging frame period Tf falls within a predetermined unit of light emission time. The same-color light source controller 144 causes the same-color laser light sources LD2 to LD5 to emit at least one pulse within the imaging frame period Tf and to increase or decrease a pulse width, performing the light control. The predetermined unit of light emission time is a minimum pulse width gradation that is a unit gradation for an increase or a decrease in a pulse width. Within the imaging frame period Tf, a difference in light emission time among the laser light sources LD2 to LD5 included in different groups corresponds to zero or the minimum pulse width gradation.

The minimum pulse width gradation is set to a time during which a stable pulse waveform can be generated in the laser light sources or the same-color light source controller 144, for example. The minimum pulse width gradation is, for example, several μs to several tens of μs.

The present modification achieves effects similar to those achieved by the first embodiment.

### Modification 3

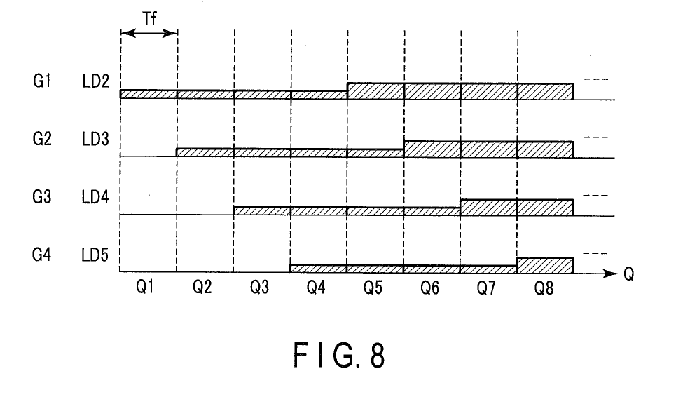

Hereinafter, yet another modification of a light control method of emitted light from the same-color laser light sources LD2 to LD5 will be described. FIG. 8 shows driving pulses of the same-color laser light sources LD2 to LD5 according to the light control method of the present modification. As in FIG. 4, FIG. 8 shows the case where the emitted-light quantity Q of all of the green laser light sources LD2 to LD5 is increased by a unit gradation.

In the present modification also, all the same-color laser light sources LD2 to LD5 are respectively divided into different groups so that, for example, the laser light sources LD2 is in the group G1, the laser light source LD3 is in the group G2, the laser light source LD4 is in the group G3, and the laser light source LD5 is in the group G4.

Also in the present modification, the same-color light source controller 144 performs the light control of the emitted light from the same-color laser light sources LD2 to LD5 by increasing or decreasing, for each of the groups, the emitted-light quantity Q from the same-color laser light sources LD2 to LD5 within a predetermined reference period. The predetermined reference period is, for example, the imaging frame period Tf of the imaging section 162. However, in the present modification, the same-color light source controller 144 performs the light control of the emitted light from the same-color laser light sources LD2 to LD5 by increasing or decreasing, for each of the groups, the peak light quantity of the same-color laser light sources LD2 to LD5 within the imaging frame period Tf.

In the state with the light quantity Q1, within the imaging frame period Tf, the laser light source LD2 in the group G1 emits light with the peak light quantity set to a minimum peak light quantity gradation.

In the next state with the light quantity Q2, within the imaging frame period Tf, the laser light source LD3 in the group G2 emits light with the peak light quantity set to the minimum peak light quantity gradation with respect to the state with the light quantity Q1.

In the next state with the light quantity Q3, within the imaging frame period Tf, the laser light source LD4 in the group G3 emits light with the peak light quantity set to the minimum peak light quantity gradation with respect to the state with the light quantity Q2.

In the next state with the light quantity Q4, within the imaging frame period Tf, the laser light source LD5 in the group G4 emits light with the peak light quantity set to the minimum peak light quantity gradation with respect to the state with the light quantity Q3.

In the next state with the light quantity Q5, within the imaging frame period Tf, the peak light quantity with the laser light source LD2 in the group G1 is increased by the minimum peak light quantity gradation with respect to the state with the light quantity Q4.

Subsequently, the peak light quantity is increased in a similar manner.

As described above, the same-color light source controller 144 controls the same-color laser light sources LD2 to LD5 so that a difference in peak light quantity among the same-color laser light sources LD2 to LD5 included in different groups within the imaging frame period Tf falls within a predetermined unit of peak light quantity. In the present modification, the predetermined unit of peak light quantity is the minimum peak light quantity gradation. The minimum peak light quantity gradation is a unit gradation for an increase or a decrease in the peak light quantity. In the present modification, a difference in peak light quantity of the laser light sources included in different groups within the imaging frame period Tf is the minimum peak light quantity gradation.

The minimum peak light quantity gradation is, for example, the light quantity corresponding to the gradation of an increase or a decrease in driving current with which the same-color light source controller 144 can perform stable control. Furthermore, for example, the minimum peak light quantity gradation corresponds to a range of the light quantity in which the light quantity can be maintained in a stable state in the same-color laser light sources LD2 to LD5. The minimum peak gradation is, for example, several hundreds of μW to 1 mW.

The present modification also achieves effects similar to those achieved by the first embodiment.

### Other Modifications

In the first embodiment and the modifications, the same-color laser light sources LD2 to LD5 are green laser light sources, but are not limited thereto. The red light source section LSR or the blue light source section LSB may include same-color laser light sources.

In the example described in the first embodiment and the modifications, light control is performed based on control of the number of pulses, control of the pulse width, or control of the peak light quantity. However, light control may be performed based on the combination of these controls. In such a case, it is preferable for the laser light sources included in different groups that within the imaging frame period Tf, a difference in light emission time correspond to a single pulse light emission time or a minimum pulse width gradation, or a difference in peak light quantity correspond to a minimum peak light quantity gradation.

In the first embodiment and the modifications, each narrow band light source comprises a laser diode, but is not limited thereto. For example, each narrow band light source may comprise LED.

In the example described in the first embodiment, blue laser light, green laser light, and red laser light are combined to generate white light. However, this example is not a limitation. For example, it may be configured that the same-color laser light sources are blue laser light sources, and the light converter includes a wavelength converting member. In such a case, illumination light is generated by mixed transmitted light of blue laser light with wavelength converted light generated by the wavelength converting member.

In the example described in the first embodiment and the modifications, the illuminating device 140 of the present invention is applied to the endoscope including the imaging device 160. However, this example is not a limitation. The illuminating device 140 of the present invention may be applied to other observation devices including the imaging device 160, such as a microscope, for example. The illuminating device 140 of the present invention may also be applied to, for example, a projection device such as a projector not including the imaging section 162. In the case of not including the imaging section 162, the imaging frame period Tf may be replaced, as a reference period, with a period shorter than the period of the reciprocal of the critical fusion frequency (a frequency above which a human eye can not recognize a change in brightness and darkness). Since the critical fusion frequency is generally said to be approximately 30 to 50 Hz, for example, 20 ms that is the reciprocal of 50 Hz is set as a reference period.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An illuminating device comprising:
narrow band light sources including same-color narrow band light sources each configured to emit same-color narrow band light, the same-color narrow band light sources being divided into groups each including at least one narrow band light source; and
a light source controller configured to control the narrow band light sources, the light source controller including a same-color light source controller configured to control the same-color narrow band light sources and perform light control of emitted light from the same-color narrow band light sources by increasing or decreasing, for each of the groups, an emitted-light quantity of the same-color narrow band light sources within a predetermined reference period, by a unit gradation in a predetermined order.

2. The illuminating device according to claim 1, wherein the same-color light source controller performs the light control of the emitted light from the same-color narrow band light sources by increasing or decreasing, for each of the groups, light emission times of the same-color narrow band light sources within the predetermined reference period, by a unit gradation in a predetermined order.

3. The illuminating device according to claim 1, wherein the same-color light source controller controls the same-color narrow band light sources so that a difference in light emission time between the same-color narrow band light sources included in different groups within the predetermined reference period falls within a predetermined unit of light emission time.

4. The illuminating device according to claim 3,
wherein the same-color light source controller causes the same-color narrow band light sources to emit at least one pulse within the predetermined reference period and to increase or decrease the number of pulses, performing the light control, and
wherein the predetermined unit of light emission time is a single pulse light emission time as a light emission time for the one pulse.

5. The illuminating device according to claim 3,
wherein the same-color light source controller causes the same-color narrow band light sources to emit at least one pulse within the predetermined reference period and to increase or decrease a pulse width, performing the light control, and
wherein the predetermined unit of light emission time is a minimum pulse width gradation as a unit gradation for an increase or a decrease in the pulse width.

6. The illuminating device according to claim 1, wherein the same-color light source controller performs the light control of the emitted light from the same-color narrow band light sources by increasing or decreasing, for each of the groups, a peak light quantity of the same-color narrow band light sources within the reference period, by a unit gradation in a predetermined order.

7. The illuminating device according to claim 6, wherein the same-color light source controller controls the same-color narrow band light sources so that a difference in peak light quantity between the same-color narrow band light sources included in different groups within the predetermined reference period falls within a predetermined unit of peak light quantity.

8. The illuminating device according to claim 7, wherein the predetermined unit of peak light quantity is a minimum light quantity gradation as a unit gradation for an increase or a decrease in a peak light quantity.

9. The illuminating device according to claim 1, wherein a difference in number between sets of same-color narrow band light sources, each of the sets being included in a different group, is one or less.

10. The illuminating device according to claim 1, wherein same-color narrow band light sources are included in a same group, and the same-color light source controller controls the same-color narrow band light sources included in the same group so that the same-color narrow band light sources included in the same group are substantially equal in emitted-light quantity.

11. The illuminating device according to claim 1, wherein the same-color light source controller causes the same-color narrow band light sources to emit at least one pulse within the predetermined reference period, and the same-color narrow band light sources are substantially equal in peak light quantity of the pulse.

12. The illuminating device according to claim 1, wherein same-color narrow band light sources are included in a same group, and the same-color light source controller performs control by synchronizing emission timings of the same-color narrow band light sources included in the same group.

13. The illuminating device according to claim 12, wherein the same-color light source controller performs control by supplying driving currents to the same-color narrow band light sources, and the same-color narrow band light sources included in the same group are connected in series.

14. The illuminating device according to claim 1, further comprising a light combiner configured to combine the same-color narrow band light emitted from the same-color narrow band light sources, wherein the light combined by the light combiner is emitted as illumination light.

15. The illuminating device according to claim 1, wherein the same-color narrow band light comprises narrow band light included in a same color range for three color ranges including a blue range, a green range, and a red range.

16. An endoscope comprising:
the illuminating device according to claim 1; and
an imaging section configured to acquire an optical image of an observation object illuminated by the illuminating device.

17. The endoscope according to claim 16, wherein the predetermined reference period is an imaging frame period of the imaging section.

18. The endoscope according to claim 17,
wherein the imaging section includes at least one kind of color filters on a front face of a light receiver, and
wherein the same-color narrow band light comprises rays of narrow band light included in a wavelength range equal to or higher than a predetermined transmittance in each of the color filters.

19. The endoscope according to claim 17, wherein the same-color narrow band light used for illumination of specific light observation in which a specific substance is emphasized and observed by illuminating illumination light in a specific wavelength range is narrow band light included in a wavelength range necessary for the specific light observation.

20. The endoscope according to claim 17, wherein same-color narrow band light used for illumination of fluorescent observation that irradiates a specific substance with excitation light to observe fluorescent light emitted from the specific substance is narrow band light included in an excitation wavelength band of the specific substance.
